(19) United States
(12) Patent Application Publication
(10) Pub. No.: US 2002/0055164 A1
(43) Pub. Date: May 9, 2002

(54) Process to create solid bodies, and uses for such bodies

(76) Inventor: Matthias Kleespies, Westerheim (DE)

Correspondence Address: COLLARD & ROE, P.C., 1077 Northern Boulevard, Roslyn, NY, 11576-1696, US

(21) Appl. No.: 09/915,469

(22) Filed: May 21, 2001

Related U.S. Application Data
(63) Continuation-in-part of application No. 09/254,827, filed on Mar. 12, 1999, now abandoned.
Application No. 09/254,827 is a 371 of international application No. PCT/DE97/01788, filed on Aug. 19, 1997.

(30) Foreign Application Priority Data
Sep. 12, 1996 (DE) 196 37 112.0

(51) Int. Cl. C12N 1/00; A23C 1/06

(52) U.S. Cl. 435/243; 426/385

(57) ABSTRACT

Nutrients are added to water kefir grains, and the arising biopolymer is mechanically harvested and dried to yield solid particles that are suitable for use as packaging, soil improver, food or feed, as bedding for animals, as the stationary phase in chromatography, and as a desiccant. The porosity of the particles is increased by freeze-drying or by adding propellants. Chemically or otherwise modified biopolymer may be used for various applications.

### CROSS REFERENCE TO RELATED APPLICATIONS

This is a continuation-in-part of U.S. patent application Ser. No. 09/254,827, filed on Mar. 12, 1999 and benefit is claimed under 35 U.S.C.§120. Benefit is also claimed under 35 U.S.C. §120 of PCT No. PCT/DE97/01788 filed Aug. 19, 1997 and 35 U.S.C. §119 of German Application No. 196 37 112.0 filed Sep. 12, 1996.

### BACKGROUND OF THE INVENTION

1. Field of the Invention

The invention concerns a process to create solid bodies from a biopolymer.

2. The Prior Art

Various procedures exist for manufacturing solid bodies and it depends on the starting material and intended use for the manufactured body.

The invention provides another procedure for manufacturing solid bodies having a starting material that is easy to select and obtain and can yield products for most any use.

### SUMMARY OF THE INVENTION

According to the present invention, solid bodies are manufactured by adding nutrients to water kefir cultures and drying the arising material.

Water kefir cultures are also described as Japanese sea crystals. They form whitish, translucent, cartilaginous clumps of irregular shapes and sizes. The clumps have an average diameter of one centimeter and are crystal clear under a microscope.

Water kefir is different from milk kefir. Milk kefir grows in milk or whey, whereas water kefir thrives in normal tap water to which sugar and nutrients have been added. Only water kefir forms the familiar cartilaginous clumps that settle in the nutrient liquid. This water kefir is known from the manufacture of different beverages. Water kefir is a community of microorganisms that includes various lactic bacteria and different kinds of non pathogenic yeasts.

Water kefir cultures that can be obtained as kefir grains are a mixture of homo and hetero-fermenting lactobacillus types, and mesophile streptococcus lactis and carbon-dioxide-producing and alcohol-producing yeasts, as well as acetobacteria. Frequently, however, only a mixture of streptococcus lactis and types of lactobacillus are used as the kefir culture. The microorganisms of the kefir culture as well as the kefir grains double in approximately two days.

According to the invention, the water kefir cultures are fermented, and the arising material which comprises a polysaccharide, which is then dried to yield a solid body.

Water kefir has an advantage that it forms water-insoluble biopolymer grain comprised of polysaccharides. The down-stream processing required to obtain the biopolymer is therefore greatly simplified since the biopolymer can simply be mechanically removed from the solution, especially by sieving, and it does not have to be extracted from the fermentation broth or aqueous phase.

Depending on the used nutrient composition, such additives as nitrogen, phosphate, vitamins or acidifiers are added to the polysaccharide-forming microorganisms to provide an optimum nutrient supply. The polysaccharide formation rate can also be increased by setting an optimum pH and increasing the temperature to 25-37° C.

The best methods of drying are air drying, spray drying and vacuum drying. The drying procedure must be adapted to the desired use of the product. Depending on the used drying procedure, particles, panels, or bodies dried in molds can be created. In addition, the particle sizes of the bodies can be adjusted by further treatment such as grinding.

One particular advantage of the created bodies is that they are biodegradable which is particularly favorable for many applications.

### DETAILED DESCRIPTION OF THE PREFERRED EMBODIMENT

In one advantageous embodiment of the procedure, waste is used as the nutrient. Suitable waste is residue from the sugar industry and sugar-processing industry, or residue from starch manufacturing or processing which produces a large amount of sugar or starch. Waste from the paper industry is also suitable as a nutrient if respective enzymes or other microorganisms are added that ensure decomposition of the cellulose or starch into mono or disaccharides.

The form of the arising bodies can be varied by pouring the material into molds before it is dried. In addition, the predried mass can be extruded in extruders. This allows to adapt the particle size obtained to the desired application.

An advantageous drying process is freeze drying. If the material is freeze dried, essentially same-sized particles arise that have a porous inner structure. The increase of the volume of a set amount of material makes the bodies useful for applications in which porosity is useful.

The porosity of the manufactured bodies can also be increased by adding propellants to the material. Propellants are materials that develop propellant gases under the effect of heat or chemicals. Expanding agents are very useful (that e.g. are used in the plastics and construction materials industry to manufacture foams), and leavening agents used in the baking industry to leaven dough.

The manufactured material can also be modified chemically. E.g. spinnable biopolymers can be manufactured from the cartilaginous material and the dried material, ionic dissociatable groups can be attached, and molecules can be cross-linked or split. The liquid or solid chemically-modified material can be used in hair-care agents.

The described bodies can be used for various applications. A particularly important one is packaging or as packaging aids. They can replace previously-used packaging or packaging aids such as chips, hollow spheres or hemispheres or in a shape adapted to the product. The advantage is that the bodies according to the invention can be composted with the other packaging, or they are at least biodegradable. Recycling the packaging or packaging aids is greatly simplified by using the bodies according to the invention.

Another important application of the bodies according to the invention is their use as soil improver. The bodies according to the invention can serve as particles to loosen soil, and they increase the ability of the soil to bind and store water.

Depending on the used starting materials, the bodies according to the invention can be used as food or feed. The bodies can be mixed with conventional feeds. Both the consistency and the composition of the bodies makes them useful as concentrated feed for cows and pigs.

The bodies are also quite suitable to be used as a desiccant or absorbent since they are particularly hygroscopic due to their composition and physical structure. In particular, finely grinding the bodies produces a superabsorbent that is outstanding for making biodegradable diapers.

Another application for the bodies is in the construction industry where they can be used as insulation in particle form or as panels and molded bodies.

In addition, the bodies in the form of particles are suitable as bedding for animals, and particularly as cat litter.

The use of the solid bodies as a solid electrolyte or as an electrolyte in an aqueous solution has many applications e.g. for sensors, batteries, fuel cells and electronic circuits.

Finally, the manufactured bodies can be used as a stationary phase in chromatography, especially in HPLC.

Liquid, sugar-containing residues from the sugar industry are added to large containers holding water kefir. The pH is set to 5, the suitable temperature is set, and then nitrogen, phosphate and vitamins are also added to ensure optimum growth conditions for the microorganisms. As the microorganisms double every two days, additional grains made up of polysaccharide arise that collect on the container floor as well and are then separated from the liquid by sieving and subsequently are dried or freeze dried. The liquid phase (fermentation broth) can be used as animal feed after drying. This means that no additional energy is necessary for biopolymer harvesting, no waste material emerges from the whole process and no waste water cleaning is necessary. Ecologically balancing the process makes it superior to any other industrial process since waste material is used as nutrients for the microorganisms and thus for the biopolymer grains, and the waste emerging from the fermentation process may be used as nutrients for animals. Also, the process requires minimal energy, since no stirring or oxygen control is necessary.

After freeze drying, porous grains arise with an approximate diameter of 4-5 mm that then can be used e.g. to enrich soil, as food or feed or as a desiccant. The larger particles are above all suitable as packaging or packaging aids to pack sensitive goods.

Accordingly, while only a few embodiments of the present invention have been shown and described, it is obvious that many changes and modifications may be made thereunto without departing from the spirit and scope of the invention.

In the claims:

1. A process for manufacturing solid bodies, comprising the steps of adding nutrients to water kefir cultures creating a biopolymer mixture; and drying said mixture to form solid bodies made of water insoluble dextrans for simplifying removal of the solid bodies from the mixture.

2. The process according to claim 1, wherein said nutrients are waste.

3. The process according to claim 1, further comprising the step of pouring the mixture into molds before drying.

4. The process according to claim 1, wherein said drying step comprises freeze drying the mixture.

5. The process according to claim 1, further comprising the step of adding a propellant to the mixture before said drying step.

6. The process according to claim 1, further comprising the step of chemically modifying the mixture before said drying step.
